(19) United States
(12) Patent Application Publication
(10) Pub. No.: US 2019/0133905 A1
(43) Pub. Date: May 9, 2019

(54) PERSONAL CLEANSING COMPOSITION

(71) Applicant: PZ CUSSONS (INTERNATIONAL) LTD, Manchester (GB)

(72) Inventors: Paul Heffernan, Oakmere (GB); Phillip Shaw, Bowdon (GB); Meiki Wu, Manchester (GB); Craig Davies, Alsager (GB)

(73) Assignee: PZ CUSSONS (INTERNATIONAL) LTD, Manchester (GB)

(21) Appl. No.: 16/096,887

(22) PCT Filed: Mar. 16, 2017
(86) PCT No.: PCT/GB2017/050724

§ 371 Date: Oct. 26, 2018

(30) Foreign Application Priority Data
Apr. 26, 2016 (GB) 1607197.9

(51) Int. Cl. A61K 8/41; A61K 8/46; A61K 8/81; A61K 8/04; A61Q 5/02; A61Q 19/10

(52) U.S. Cl. CPC A61K 8/41; A61K 8/463; A61K 8/8152; A61K 8/042; A61K 2800/28; A61Q 19/10; A61K 2800/546; A61K 2800/596; A61Q 5/02

(57) ABSTRACT

The present invention relates to personal cleansing compositions in liquid or gel form, which can be used for cleansing the skin and hair and is particularly useful as a body wash, facial wash or hand wash. A key feature of the invention is its highly structured nature arising from interaction of polymeric and surfactant constituents, which is useful for the stable incorporation of insoluble benefit agents such as particulate exfoliants, encapsulates or oils. Furthermore, the present invention is cost effective, offers a high degree of mildness, has desirable in-use properties and is easy to manufacture.

### CROSS REFERENCE TO RELATED APPLICATIONS

This application is a National Stage application of PCT/GB2017/050724, filed Mar. 16, 2017, which claims the benefit of Great Britain Application No. 1607197.9, filed Apr. 26, 2016, both of which are incorporated by reference in their entirety herein.

### FIELD

The present invention relates to personal cleansing compositions in liquid or gel forms, which have a highly structured nature and are thus able to incorporate insoluble benefit agents such as particulate exfoliants, encapsulates or oils. These new compositions are cost effective, offer a high degree of mildness, have desirable in-use properties and are easy to manufacture.

### BACKGROUND

Personal cleansing compositions that are unstructured in nature are incapable of suspending insoluble materials such as hydrophobic oils or waxes, scrub particles (natural or synthetic) or encapsulates (containing e.g. fragrance oils, moisturising oils or pigments). Such insoluble materials will tend either to sink, if their density is greater than a base matrix of the composition, or rise upwards towards the surface if their density is lower than the base matrix. As a consequence of this, such personal cleansing compositions have unacceptable levels of storage stability.

This problem is well understood in the art, and various solutions have previously been described and commercialised. Essentially, the solutions fall into two broad camps:
A) Use ‘external’ structurants within the aqueous phase of the product. These external structurants include natural gums such as xanthan and guar derivatives, and synthetic polymers such as carbomers and acrylate derivatives and clays (natural or synthetic). These materials are sometimes used in combination; or
B) Use structured surfactant systems. These rely on aggregation structures formed by the surfactants, typically lamellar in nature, which can either be in the form of parallel sheets or multi-lamellar vesicles known as spherulites. Such systems require precise combinations of particular surfactants with the right physical shape, and also sometimes require an inducer which may be a simple electrolyte such as sodium chloride or a fatty acid or fatty alcohol.

Examples of prior compositions which fall under type (A):

US 2004/0121925 describes a composition with dual gum system, comprising a stable, clear aqueous cleansing phase having stably dispersed and suspended therein particles bearing conditioning agents. The suspending system is based on a combination of xanthan gum (about 0.4-1.5%) and guar gum (about 0.2-1.5%), and has a clarity of less than or equal to 20 Nephelometric Turbidity Units (NTU).

U.S. Pat. No. 6,001,344 describes a novel structuring system comprising both xanthan gum and cross-linked polyacrylic acid polymer, which combination is said to provide enhanced stability for large size benefit agent droplets in an amphoteric/anionic surfactant system relative to either structurant alone.

U.S. Pat. No. 6,846,785 describes a liquid soap having vitamin containing microcapsules including a base have an anionic surfactant and a chelating agent, a cross-linked acrylic polymer suspending agent and multiple vitamin-containing microcapsules uniformly suspended in the liquid soap. The liquid soap is formulated at an elevated temperature which is maintained throughout the formulation process.

Examples of prior compositions which fall under type (B):

U.S. Pat. No. 7,488,707 describes an aqueous structured surfactant system, containing water, one or more anionic surfactants, and one or more alkoxylated nonionic surfactants. It is said to exhibit shear-thinning viscosity, be capable of suspending water insoluble or partially water soluble components, and be mild to the eyes and skin.

US 2008/0233061 describes a structured surfactant composition, comprising i) from greater than 0 to 15 parts by weight of an alkyl ether sulfate surfactant, ii) from greater than 0 to 15 parts by weight of an alkyl sulfate surfactant, iii) from greater than 0 to 8 parts by weight of an alkanolamide surfactant, iv) from 0 to 10 parts by weight of an amphoteric surfactant, providing that the total amount of components (i), (ii), (iii) and (iv) is greater than or equal to 5 parts by weight, v) an amount of electrolyte effective in combination with (i), (ii), (iii) and (iv), providing a structured surfactant composition having an opaque visual appearance and exhibiting a yield strength of greater than 0 Pascals, and (vi) water.

U.S. Pat. No. 5,556,628 describes storage-stable, pseudoplastic free-flowing cosmetic compositions/formulations, such as shampoos, shower and exfoliating gels and hair lotions, comprising a stable and homogeneous suspension in water of water-insoluble particulates, and which further comprise at least one anionic surfactant, at least one nonionic or amphoteric surfactant and at least one electrolyte, these surfactants being present in such amounts as to impart pseudoplasticity thereto with a yield point of at least 0.2 Pa and constituting spherulites suspended within a lamellar phase.

U.S. Pat. No. 5,952,286 describes lamellar phase compositions comprising defined surfactant systems and a structurant selected from liquid fatty acids, liquid alcohols and derivatives thereof, where the structurant is responsible for inducing the lamellar phase.

WO 2009/063250 describes an aqueous shear-thinning personal care composition comprising: a) at least 3% of a sulfonate surfactant of general formula (I):

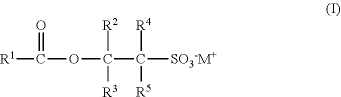

b) at least 3% of an amphoteric or zwitterionic surfactant; c) at least 10% of water; and d) at least 0.5% of an additional component in the form of particles or droplets suspended in the composition.

Examples of prior compositions which are based on elements of both approaches include the following:

WO 2011/057909 describes a mild, substantially isotropic skin cleansing solution able to suspend insoluble components and produce lather. The cleanser is formulated with synthetic anionic surfactants and a specific ratio of carboxylic acid(s) to hydrophobically modified cross-linked acrylate copolymer(s). The carboxylic acid and acrylate polymer combination were said to provide a synergistic effect on zero shear viscosity at 25° C. in a specific pH and copolymer/acid concentration ratio range.

While these approaches can be effective at providing the necessary rheology to suspend insoluble/partially soluble matter, they suffer from a number of disadvantages.

Products structured with natural gums, such as those described in US 2004/0121925, have undesirable consumer rheology, appearing thick at rest but unacceptably runny in use, and are perceived as being inconsistent or ‘gloopy’.

Products that use synthetic polymers are considered to have much better flow behaviour, but are typically expensive due to the high cost and inclusion level of such materials. Some polymers, such as carbomers, are also very sensitive to electrolytes and surfactants, which reduces their effectiveness.

Compositions based on structured surfactant systems typically require specialised blends of surfactants which are required to be added at a high level of inclusion, and have only limited flexibility in terms of their ability to include other types of surfactants or benefit agents, as these may affect the overall ‘structurisation’. The desired product texture usually requires the incorporation of high levels of oil (typically 15-50%). While in some cases this may be desirable, it significantly increases the formulation or finished product costs. It would be more desirable to have the flexibility and freedom to vary the oil level from 0 to 50%, without the prohibitive cost implications.

A further disadvantage is that compositions based on structured surfactant systems tend to have an opaque visual appearance, making them unsuitable for manufacturing clear gels. Further, in some cases, hot processes are required to melt solid ingredients such as fatty acids; cold mixing is preferable as this shortens production time as no heating and cooling steps are required, and also reduces energy requirements, and thus the overall cost of production.

There is therefore a need for a personal cleansing composition which is able to overcome one or more of the above problems, and incorporate insoluble benefit agents such as particulate exfoliants, encapsulates or oils in a stable manner, has a longer shelf life, which is cost effective, and is easy to manufacture.

### SUMMARY

Therefore, in accordance with the present invention there is provided a personal cleansing composition comprising:
a) water;
b) one or more anionic surfactants in an amount of about 2 to about 20% by weight of the composition;
c) one or more amine oxide surfactants in an amount of about 1 to about 10% by weight of the composition; and
d) a hydrophobically modified alkali-swellable acrylic emulsion (HASE) polymer in an amount of about 0.5 to about 3% by weight (as polymer solids) of the total composition;
   wherein the composition is capable of suspending water insoluble or partially water soluble substances in the form of particles or liquid droplets.

The concept of ‘alkali-swellable’ polymers is well known in the polymer technical field, and as such will be familiar to, and understood by, a person skilled in the art. Such HASE polymers are also commercially available, for example under the ACULYN® trade name from Dow Chemicals, or under the Rheovis® and Latekoll® trade names from BASF.

In their commercially available forms, the polymers (which are acidic in their nature) are coiled up tightly and dispersed in an aqueous medium, essentially as insoluble particles. In the presence of an alkali, the acidic polymer is neutralized, and expands due to negative charge repulsion. The particles of polymer are therefore said to swell, with water filling up the spaces. Not all synthetic anionic polymers swell in the presence of alkalis, for example the type used as opacifying agents (e.g. styrene/acrylates copolymer) are designed to remain in a compact state.

When HASE polymers/rheology modifiers are neutralized, they become anionically charged and water-soluble. When HASE polymers dissolve, they swell due to charge-to-charge repulsion and thicken instantly. As in the case of cross-linked alkali-swellable emulsion (ASE) polymers, structure is built as the critical concentration is exceeded, but in the case of the HASE rheology modifiers, it is by polymer chain entanglement, in conjunction with association of the extended hydrophobic groups on the polymer backbone with each other and other hydrophobic groups in a formulation.

The solution to the problem detailed above is rheological in nature. A key feature of the invention is its highly structured nature arising from interaction of polymeric and surfactant constituents. This structuring is useful for the stable incorporation of insoluble benefit agents such as particulate exfoliants, encapsulates or oils.

In general terms, it is necessary to provide a sufficiently high viscosity under zero shear conditions, in order to prevent movement of particles and/or oil droplets when the formulation is at rest; and it is also necessary also for the viscosity of the composition to drop substantially when the product is exposed to shear forces, e.g. during dispensing from final packaging and spreading onto a person's skin or hair during use.

This effect is achieved by the present invention. Under zero shear conditions, the composition is able to maintain a sufficiently high viscosity to ensure formulation stability at rest, but under moderate shear conditions, the viscosity falls significantly, allowing for the release of the incorporated components such as exfoliants or oils, that a consumer wants to have in contact with their skin during cleansing.

In the present invention, this effect is achieved by the use of a HASE polymer, in combination with both of anionic and amine oxide surfactants. The benefits of such a combination has not before been taught in the prior art.

### DETAILED DESCRIPTION

The present invention uses the type (A) approach detailed hereinabove, enhanced by a specific surfactant interaction which, while not wishing to be bound by any particular theory, is explained below:

The inventors have found that mixing a pH neutral solution of an anionic surfactant with a pH neutral solution of an amine oxide produces an unexpected rise in pH of the aqueous medium, from about 7.0 to about 9.0 Amine oxides are nonionic surfactants at neutral pH but with a strong dipole across the N—O bond. It is reasonable to hypothesise that this strong dipole is capable of interacting with anionic surfactants and surrounding water molecules which may account for the observed pH rise. Further, the inventors have found that inclusion of a particular type of polymer (Hydrophobically modified Alkali Swellable Emulsion polymer, herein referred to as HASE polymer for short) produces a 3-way interaction between the polymer and the two surfactant types which gives an unexpectedly large increase in viscosity.

This effect is exploited in the present invention in two ways. Firstly, the rise in pH from the surfactant interaction described earlier can be used to neutralise the acidic HASE polymer which would otherwise require the use of a basic neutralising agent such as sodium hydroxide. Secondly, it has been found that the resulting anionic-amine oxide complexation interacts strongly with the HASE polymer. This interaction therefore allows the use of a lower polymer level than normally would be required to achieve the very high zero shear viscosity required to enable the suspension of particulates and oil droplets.

This is believed to be a new type of structurisation. It is not believed that the surfactant interaction creates lamellar structures, as is described in the prior art (approach B detailed above).

It is believed that the pairing of the two surfactant molecules creates a more hydrophobic (and larger) entity that interacts more strongly with the hydrophobic appendages on the HASE polymer, building a stronger surfactant-polymer network.

It is also believed that the pairing of the surfactant molecules in this manner has the potential to reduce irritancy to a person's skin, relative to using the anionic surfactant alone, by: a) reducing the amount of free monomers in solution; b) the anionic charge neutralisation reducing its binding affinity to skin proteins; and c) the larger paired surfactant entities are less able to penetrate the skin. Evidence of this is provided in Surfactants in Cosmetics (Surfactant Science Series volume 68, published by CRC Press), 2^{nd} edition, page 405, under the heading “Surfactant interactions that counter skin irritation”, where data is provided using an in-vitro based method based on swelling of isolated stratum corneum tissue. This shows that the degree of swelling, as measured by % increase in hydrated length, was reduced from 46.1% (1% sodium lauryl sulfate (SLS) only) to just 7.7% for a solution containing 1% SLS+1% lauryldimethylamine oxide (LDAO). The result for a solution of 1% SLS+1% cocamidopropyl betaine (a commonly used amphoteric surfactant well known for reducing irritancy of anionics when used in combination) was 21.0%, a significant reduction but not as dramatic as in the case of LDAO.

Furthermore, the inventors have also found that the resulting compositions have advantageous sensory qualities when used for personal washing, in particular producing a voluminous yet creamy lather and a pleasant skin-feel during the washing process which persists as a conditioned feel after washing.

A further advantage of this novel approach is that it uses common surfactants, which is cost-effective. The resulting formulations have a desirably thick but shear-thinning gel texture. This texture is not dependent on the presence of oils, but is flexible in its ability to allow the stable incorporation of oils from zero up to at least 50%. It is also easy to manufacture, has good lathering characteristics, and has been found to leave the skin feeling softer and smoother.

### HASE Polymer Constituent:

HASE polymers or thickeners are well known in the art, and are the associative analogue to ASE synthetic thickeners which are produced commercially by emulsion polymerisation. Like the ASE thickeners, HASE polymers have ‘backbones’ made from methacrylic acid and various acrylate/methacrylate alkyl ester monomers. However, unlike typical ASE types, HASE polymers contain hydrophobic “pendants” which are commonly attached via polyethylene oxide chains. Thickening is achieved by 2 mechanisms: firstly, upon addition of an alkaline neutralising base (for example an alkali metal hydroxide), the —COOH moieties along the backbone become ionised to —COO⁻ carboxyl groups which cause the polymer chain to expand by electrostatic repulsion and become soluble in water, changing from a milky low viscosity dispersion to a transparent gel. Secondly, the hydrophobic pendant groups are able to build associations with each other and also with surfactants, oil droplets and any other dispersed particulates. This creates a network structure that is accompanied by significant viscosity build.

Suitable HASE polymers for use in the present invention are anionic in nature upon neutralisation. They are typically supplied in the form of an aqueous liquid emulsion (which has an un-neutralised, acidic pH) form with hydrophobic appendages which make them ‘associative’—i.e. they can associate with other formulation ingredients, such as surfactants, oils or particles.

Suitable HASE polymers include, but are not limited to, those available from Rohm & Haas under the Aculyn trade name, such as Aculyn 22 (acrylates/steareth-20 methacrylate copolymer), Aculyn 28 (acrylates/beheneth-25 methacrylate copolymer) and Aculyn 88 (acrylates/steareth-20 methacrylate crosspolymer). Another option is Synthalen W2000 from 3V Sigma (acrylates/palmeth-25 acrylates crosspolymer) and Novethix L-10 Polymer (acrylates/beheneth-25 methacrylate copolymer) from Lubrizol.

The HASE polymer is present in an amount of about 0.5 to about 3% by weight (as polymer solids) of the total composition, typically from about 1 to about 2% by weight.

### Anionic Surfactant Constituent:

Suitable anionic surfactants for use in the composition of the invention include, but are not limited to, alkyl sulfates (e.g. sodium lauryl sulfate or sodium cocosulfate); ethoxylated alkyl sulfates (e.g. sodium laureth sulfate or sodium trideceth sulfate); alpha olefin sulfonates; alkyl alkoxy carboxylates; acyl isethionates (e.g. sodium cocoyl isethionate); acyl sarcosinates (e.g. sodium cocoyl sarcosinate); sulfosuccinates (e.g. disodium laureth sulfosuccinate); or acyl glutamates such as sodium cocoyl glutamate. Alternative counter ions may be used in place of sodium, such as potassium, ammonium, triethanolammonium (TEA), or monoisopropylammonium (MIPA). According to one embodiment, the anionic surfactant system includes, or is, sodium laureth sulfate.

The one or more anionic surfactants are present in an amount of about 2 to about 20% by weight of the composition; typically from about 5 to about 15% by weight, more typically from about 6 to about 12% by weight, more typically from about 7 to about 10% by weight.

### Amine Oxide Constituent:

Suitable amine oxide surfactants for use in the composition of the invention include, but are not limited to, alkamine oxides containing at least one long hydrocarbon chain containing at least eight carbon atoms.

One such class of amine oxides is the alkyl di(lower alkyl) amine oxides, wherein the alkyl group therein contains from 8 to 22, and preferably about 10 to about 16, carbon atoms, and can be straight or branched chain, saturated or unsaturated. The lower alkyl groups in the alkyl di(lower alkyl) amine oxides contain from 1 to 7 carbon atoms, and typically are methyl, ethyl, propyl or butyl, more typically methyl. Specific examples include, but are not limited to, lauryl dimethyl amine oxide, myristyl dimethyl amine oxide, dimethyl cocoamine oxide, dimethyl (hydrogenated tallow) amine oxide, myristyl/palmityl dimethyl amine oxide, myristyl/lauryl dimethyl amine oxide, cetyl dimethyl amine oxide, stearyl dimethyl amine oxide, and myristyl/cetyl dimethyl amine oxide. These alkamine oxides have a general structural formula:

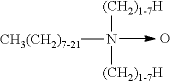

Another class of useful amine oxides for use in the present invention includes alkyl di(hydroxy lower alkyl) amine oxides in which the alkyl group contains from 8 to 22, and preferably about 10 to about 16 carbon atoms, and can be straight or branched chain, saturated or unsaturated. The lower alkyl groups in the alkyl di(lower alkyl) amine oxides contain from 1 to 7 carbon atoms, and typically are methyl, ethyl, propyl or butyl, more typically methyl or ethyl. Specific examples, include, but are not limited to, bis(2-hydroxyethyl) cocoamine oxide, and bis(2-hydroxyethyl) stearylamine oxide. These alkamine oxides have a general structural formula:

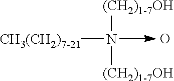

Additional useful amine oxides for use in the present invention are termed alkamidopropyl di(lower alkyl) amine oxides in which the alkyl group contains from 8 to 22, and preferably about 10 to about 16 carbon atoms, and can be straight or branched chain, saturated or unsaturated. The lower alkyl groups in the alkyl di(lower alkyl) amine oxides contain from 1 to 7 carbon atoms, and typically are methyl, ethyl or propyl, more typically methyl. Non-limiting examples thereof are cocoamidopropyl dimethyl amine oxide and lauramidopropyl dimethyl amine oxide. These alkamine oxides have a general structural formula:

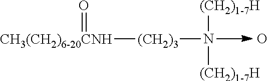

The above classes of alkamine oxide surfactants contain one or more C8-C22 alkyl groups selected from, for example, octyl, decyl, undecyl, lauryl, tridecyl, myristyl, cetyl, stearyl, isostearyl, oleyl, and mixtures thereof. Non-limiting examples of amine oxide surfactants include, but are not limited to, decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germamidopropylamine oxide, isostearamido-propylamine oxide, stearamine oxide, stearamido-propylamine oxide, decylamine oxide, dihydroxyethyl C8-C18 alkoxypropylamine oxide, myristamidopropylamine oxide, myristamine oxide, palmitamidopropylamine oxide, palmitamine oxide, undecylenamidopropylamine oxide, and mixtures of any two or more thereof. Preferred alkamine oxide surfactants are the alkyl di(lower alkyl) amine oxides in which the alkyl group contains about 12 to about 16 carbon atoms, including lauramine oxide, myristamine oxide, cocamine oxide, cetamine oxide, and mixtures of any two or more thereof. According to one embodiment of the invention, the amine oxide surfactant comprises, or is, lauramine oxide.

The one or more amine oxide surfactants are present in an amount of about 0.5 to about 10% by weight of the composition; typically from about 0.75 to about 5% by weight, more typically from about 0.9 to about 3% by weight, more typically from about 1 to about 2% by weight.

According to one embodiment of the invention, the composition of the invention may further contain one or more electrolytes, such as, for example, but not exclusively, sodium chloride. If used, the amount of electrolyte would be up to about 2 wt %.

According to another embodiment of the invention, the composition may further contain one or more of the following components:

Oils (including vegetable triglycerides, mineral oil, petrolatum, hydrocarbons, silicones, plant derived butters, esters, essential oils, compounded fragrance oils); natural or synthetic scrub particles (e.g. polyethylene, silica, rice bran, loofah, pumice, diatomaceous earth, pearlite, ground fruit or nut ‘stones’ or shells); visual effect agents (e.g. wax particles, polymeric opacifiers, inorganic ‘shimmers’); humectants (e.g. glycerol, sorbitol, glycols, urea, amino acids, lactates); cationic conditioning agents (such as cationic guar or cassia gum derivatives, Polyquaternium-7, Polyquaternium-39, Polyquaternium-10 etc.); preservatives, colorants, chelating agents and pH adjusters (either acids or bases). The formulation may optionally include a clay based rheology modifier such as a bentonite, hectorite, montmorillonite or synthetic clay such as Laponite® (commercially available from Rockwood Products) which is available in various grades.

The various components of the composition of the invention are added in their respective amounts as desired; the balance of the composition to 100% is water.

The composition of the invention may further contain one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances.

The pH of the invention is typically in the range of about 6.0 to about 10.0, and more typically in the range of about 6.0 to about 8.0. The visual appearance of the composition in the absence of suspended matter is a translucent gel.

A further embodiment of the present invention is for the stable incorporation of liquefied gases, particularly hydrocarbons. Unexpectedly, the inventors have found that, rather than increasing the turbidity of the base gel (due to dispersed hydrocarbon droplets), certain hydrocarbons when mixed into the gel actually reduce the turbidity of the system following subsequent pressurisation. This enables the making of post-foaming gels with acceptable gel transparency. It has also been found that this is possible using much lower surfactant levels, e.g. 5-10%, than those which are normally required for post-foaming gels, which typically require over 20% surfactant content to produce the necessary micro-structure to stably incorporate hydrocarbons.

Additionally, the compositions of the present invention are also cost effective to manufacture, offer a high degree of mildness which is desirable for many consumers, have desirable in-use properties, and are easy to manufacture.

Such post-foaming gel systems are suitable for use as personal cleansing compositions, such as a body wash, facial wash or hand wash, for washing hair, or as shaving gels.

Also provided in accordance with the present invention is a method of manufacturing a personal cleansing composition comprising:
a) water;
b) one or more anionic surfactants in an amount of about 2 to about 20% by weight of the composition;
c) one or more amine oxide surfactants in an amount of about 1 to about 10% by weight of the composition; and
d) a hydrophobically modified alkali-swellable acrylic emulsion (HASE) polymer in an amount of about 0.5 to about 3% by weight (as polymer solids) of the total composition;
   wherein the composition is capable of suspending water insoluble or partially water soluble substances in the form of particles or liquid droplets,
   the method comprising the steps of:
i) loading an amount of water into a suitable vessel;
ii) adding a HASE polymer, one or more anionic surfactants and one or more amine oxide surfactants sequentially in any order, with sufficient mechanical stirring to ensure that each material is fully dispersed before adding the next; and
iii) continuing mixing until a uniform consistency is achieved.

Optionally, any of the surfactant or polymer constituents may be pre-mixed with water (with an equal amount of water being subtracted from the starting an amount of water added in step (i)) in any proportion before being added.

An exemplary, but non-limiting, method in accordance with the invention comprises the following steps:
i) dissolve one or more anionic surfactants in an amount of water;
ii) add HASE polymer emulsion, mixing until the polymer is substantially dispersed in the one or more anionic surfactants;
iii) add one or more amine oxide surfactants; and
iv) mix until substantially uniform consistency is achieved.

Alternatively, depending upon the specific polymer being used, one may add the HASE polymer to an amount of water first, before the one or more anionic surfactants, then adding the one or more amine oxide surfactants and mixing until substantially uniform consistency is achieved.

Optional ingredients (e.g. fragrances, colours, preservatives and benefit agents) may be added directly into the mix either before or after any of the stages indicated above.

Also provided in accordance with the present invention is a use of a composition comprising:
a) water;
b) one or more anionic surfactants in an amount of about 2 to about 20% by weight of the composition;
c) one or more amine oxide surfactants in an amount of about 1 to about 10% by weight of the composition; and
d) a hydrophobically modified alkali-swellable acrylic emulsion (HASE) polymer in an amount of about 0.5 to about 3% by weight (as polymer solids) of the total composition;
   wherein the composition is capable of suspending water insoluble or partially water soluble substances in the form of particles or liquid droplets, as a personal cleansing composition, such as a body wash, facial wash or hand wash, for washing hair, or as shaving gels.

The present invention will be further described with reference to the following examples, which are intended to be illustrative only, and in no way limiting upon the scope of the invention.

### EXAMPLES

TABLE 1

| | | Formulations | | | | | |
|---|---|---|---|---|---|---|---|
| Ingredients | Note | A | B | C | D | E | F |
| Sodium Laureth Sulfate | 1 | 8.00 | 8.00 | 8.00 | 8.00 | 5.00 | 8.00 |
| Sodium Lauroyl | 2 | | | | | | |
| Sarcosinate | | | | | | | |
| Sodium Cocoyl | 3 | | | | | | |
| Isethionate | | | | | | | |
| Disodium Laureth | 4 | | | | | | |
| Sulfosuccinate | | | | | | | |
| Disodium Cocoyl | 5 | | | | | | |
| Glutamate | | | | | | | |
| Lauramine Oxide | 6 | | 2.00 | | | | |
| Lauramine/Myristamine | 7 | 2.00 | | 2.00 | | 0.96 | |
| Oxide | | | | | | | |
| Cocamine Oxide | 8 | | | | | | 2.00 |
| Lauric/Myristic | 9 | | | | 2.00 | | |
| Amidopropylamine | | | | | | | |
| Oxide | | | | | | | |
| Acrylates/Steareth-20 | 10 | 1.00 | 1.00 | 1.00 | 1.50 | 0.90 | 1.20 |
| methacrylate | | | | | | | |
| crosspolymer | | | | | | | |
| Acrylates/Palmeth-25 | 11 | | | | | | |
| acrylate crosspolymer | | | | | | | |
| Acrylates/Beheneth-25 | 12 | | | | | | |
| methacrylate copolymer | | | | | | | |
| Sodium Lithium | 13 | 0.20 | | | 0.20 | 0.20 | 0.20 |
| Magnesium Silicate | | | | | | | |
| Polyquaternium-7 | 14 | 0.27 | 0.27 | 0.27 | 0.27 | 0.27 | |
| PEG-200 Hydrogenated | 15 | | 1.00 | 1.00 | 1.00 | 1.00 | |
| Glyceryl Palmate (and) | | | | | | | |
| PEG-7 Glyceryl | | | | | | | |
| Cocoate | | | | | | | |
| Tetrasodium Glutamate | 16 | 0.10 | 0.10 | 0.10 | 0.10 | 0.10 | 0.10 |
| Diacetate | | | | | | | |
| Glycerin | 17 | 1.00 | 1.00 | 1.00 | 1.00 | 1.00 | 1.00 |
| DMDM Hydantoin | 18 | 0.20 | 0.20 | 0.20 | 0.20 | 0.20 | 0.20 |
| Fragrance Oil | 19 | 0.70 | 0.70 | 0.70 | 0.70 | 0.70 | 0.70 |
| Sunflower Oil | 20 | 10.00 | 10.00 | 5.00 | 10.00 | 3.00 | |
| Silica (scrub particles) | 21 | 1.00 | | | | | |
| Visual effect pigment | 22 | | | | | | 0.10 |
| (Mica, Titanium | | | | | | | |
| dioxide, Tine oxide | | | | | | | |
| composite) | | | | | | | |

TABLE 2

| | | Formulations | | | | | |
|---|---|---|---|---|---|---|---|
| Ingredients | Note | G | H | I | J | K | L |
| Sodium Laureth Sulfate | 1 | | 7.50 | | | | 7.50 |
| Sodium Lauroyl | 2 | | | | | 7.50 | |
| Sarcosinate | | | | | | | |
| Sodium Cocoyl | 3 | 7.50 | | | | | |
| Isethionate | | | | | | | |
| Disodium Laureth | 4 | | | 7.50 | | | |
| Sulfosuccinate | | | | | | | |
| Disodium Cocoyl | 5 | | | | 7.50 | | |
| Glutamate | | | | | | | |
| Lauramine Oxide | 6 | | | | | | |
| Lauramine/ | 7 | 2.50 | 2.50 | 2.50 | 2.50 | 2.50 | 2.50 |
| Myristamine Oxide | | | | | | | |
| Cocamine Oxide | 8 | | | | | | |
| Lauric/Myristic | 9 | | | | | | |
| Amidopropylamine | | | | | | | |
| Oxide | | | | | | | |
| Acrylates/Steareth-20 | 10 | 1.30 | | 1.30 | 1.30 | 1.30 | |
| methacrylate | | | | | | | |
| crosspolymer | | | | | | | |
| Acrylates/Palmeth-25 | 11 | | | | | | 1.30 |
| acrylate crosspolymer | | | | | | | |
| Acrylates/Beheneth-25 | 12 | | 1.30 | | | | |
| methacrylate copolymer | | | | | | | |
| Sodium Lithium | 13 | 0.40 | 0.40 | 0.40 | 0.40 | 0.40 | 0.40 |
| Magnesium Silicate | | | | | | | |
| Polyquaternium-7 | 14 | | | | | | |
| PEG-200 Hydrogenated | 15 | | | | | | |
| Glyceryl Palmate (and) | | | | | | | |
| PEG-7 Glyceryl | | | | | | | |
| Cocoate | | | | | | | |
| Tetrasodium Glutamate | 16 | 0.10 | 0.10 | 0.10 | 0.10 | 0.10 | 0.10 |
| Diacetate | | | | | | | |
| Glycerin | 17 | | | | | | |
| DMDM Hydantoin | 18 | 0.20 | 0.20 | 0.20 | 0.20 | 0.20 | 0.20 |
| Fragrance Oil | 19 | 0.79 | 0.79 | 0.79 | 0.79 | 0.79 | 0.79 |
| Sunflower Oil | 20 | | | | | | |
| Silica (scrub particles) | 21 | | | | | | |
| Visual effect pigment | 22 | | | | | | |
| (Mica, Titanium | | | | | | | |
| dioxide, Tine oxide | | | | | | | |
| composite) | | | | | | | |

### Notes

1 Steol CS 270 (Stepan Company)—supplied as 70% active paste
2 Surfacare L30 (Surfachem)—supplied as 30% active solution
3 Elfan AT84 (Akzo Nobel N.V.)—supplied as a powder
4 Rewopol SB FA 30B (Evonik Industries AG)—supplied as 30% active solution
5 Plantapon ACG HC (BASF Personal Care)—supplied as 50% active solution
6 Mackamine LO E (Solvay Europe)—supplied as 30% active solution
7 Mackamine LA (Solvay Europe)—supplied as 30% active solution
8 Mackamine CS (Solvay Europe)—supplied as 30% active solution
9 Mackamine LMDO (Solvay Europe)—supplied as 30% active solution Aculyn 88 (The Dow Chemical Company)—supplied as a 29% polymer solids
10 dispersion
11 Synthalen W2000 (3V Sigma)'supplied as a 31% polymer solids dispersion
12 Novethix L-10 (The Lubrizol Corporation)—30% polymer solids
13 Laponite XLS (BYK Additives)
14 Mackernium 007B (Solvay Europe)
15 Rewoderm LIS 80 (Evonik Industries AG)
16 Dissolvine GL38 (Akzo-Nobel N.V.)
17 Cremer Oleo
18 Microcare DH (Thor Personal Care)
19 Seven Scent
20 Food grade, acquired from retail store
21 Sorbosil BFG 50 (PQ Corporation)
22 Timiron Silk Gold (Merck Performance Materials)

In each of the formulations listed in Tables 1 and 2, the balance was made up with water. All figures refer to % w/w as active substance with the exception of Rewoderm LIS 80 for which the inclusion levels are quoted as supplied.

Examples A-E are opaque white with a cream-like texture and visual appearance.

Example F is a viscous translucent gel with an iridescent visual appearance.

Example G-L are all translucent viscous gels.

The visual appearances of the formulations of the examples varies from translucent to opaque, depending on the presence of the suspended water-insoluble substances. In contrast, the systems and formulations of the existing compositions based on structured surfactant systems always results in opaque compositions; the necessary base technology to be able to provide a clear composition is not an option in the existing systems in the field of personal cleansing products. The compositions of the invention allow a person skilled in the art the choice of whether to prepare a translucent or opaque composition.

For example, the presence of vegetable oils in the formulation generally gives the formulation an opaque appearance due to the nature in which it is dispersed. This demonstrates the ability of the formulations to suspend water insoluble or partially water soluble substances in the form of particles or liquid droplets therein. Examples A and F, on the other hand, have solid particulates suspended therein, thus demonstrating the ability of the base formulation to suspend different types of insoluble matter.

All of the formulations A-L listed above are able to act as personal cleansing compositions which are able to incorporate insoluble benefit agents in a stable manner, have long shelf lives, are cost effective, and are easy to manufacture; the formulations A-F actually contain said benefit agents (in the form of silica, oils and visual effect pigment), whereas formulations G-L do not contain any insoluble benefit agents but are able to do so if required. Additionally, these compositions have advantageous sensory qualities when used for personal washing, in particular producing a voluminous yet creamy lather and a pleasant skin-feel during the washing process which persists as a conditioned feel after washing.

It is of course to be understood that the present invention is not intended to be restricted to the foregoing example, which is described by way of illustration only.

1.-26. (canceled)

27. A personal cleansing composition comprising:
a) water;
b) one or more anionic surfactants in an amount of about 2 to about 20% by weight of the composition;
c) one or more amine oxide surfactants in an amount of about 1 to about 10% by weight of the composition; and
d) a hydrophobically modified alkali-swellable acrylic emulsion (HASE) polymer in an amount of about 0.5 to about 3% by weight (as polymer solids) of the total composition;
wherein the composition is capable of suspending water insoluble or partially water soluble substances in the form of particles or liquid droplets.

28. A composition according to claim 27, wherein the HASE polymer is provided in the form of an aqueous liquid emulsion.

29. A composition according to claim 27, wherein the HASE polymer is selected from an acrylate/steareth-20 methacrylate copolymer, an acrylate/beheneth-25 methacrylate copolymer, an acrylate/steareth-20 methacrylate crosspolymer, and an acrylate/palmeth-25 acrylate crosspolymer.

30. A composition according to claim 27, wherein the HASE polymer is present in an amount of about 1 to about 2% by weight of the composition.

31. A composition according to claim 27, wherein the one or more anionic surfactants comprise one or more selected from alkyl sulfates; ethoxylated alkyl sulfates; alpha olefin sulfonates; alkyl alkoxy carboxylates; acyl isethionates; acyl sarcosinates; sulfosuccinates; or acyl glutamates;
wherein the one or more anionic surfactants optionally comprise one or more selected from sodium lauryl sulfate, sodium cocosulfate, sodium laureth sulfate, sodium trideceth sulfate, sodium cocoyl isethionate, sodium cocoyl sarcosinate, disodium laureth sulfosuccinate, or sodium cocoyl glutamate.

32. A composition according to claim 31, wherein the one or more anionic surfactants comprise sodium laureth sulfate.

33. A composition according to claim 27, wherein the one or more anionic surfactants are present in an amount of about 5 to about 15% by weight of the composition.

34. A composition according to claim 27, wherein the one or more amine oxide surfactants comprise alkamine oxides and alkylamidopropyl amine oxides containing at least one hydrocarbon chain containing at least eight carbon atoms.

35. A composition according to claim 34, wherein the one or more amine oxide surfactants comprise one or more selected from alkyl di(lower alkyl) amine oxides of the formula: 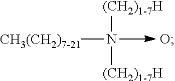
or
alkyl di(hydroxy lower alkyl) amine oxides of the formula: 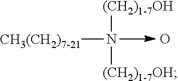
or
alkamidopropyl di(lower alkyl) amine oxides of the formula: 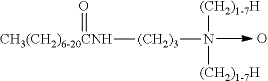
wherein the alkyl groups are straight or branched chain, saturated or unsaturated and contain from 8 to about 22 carbon atoms and the lower alkyl groups contain from 1 to 7 carbon atoms.

36. A composition according to claim 34, wherein the alkyl di(lower alkyl) amine oxides comprise one or more selected from lauryl dimethyl amine oxide, myristyl dimethyl amine oxide, dimethyl cocoamine oxide, myristyl/palmityl dimethyl amine oxide, myristyl/lauryl dimethyl amine oxide, cetyl dimethyl amine oxide, stearyl dimethyl amine oxide, and myristyl/cetyl dimethyl amine oxide;
the alkyl di(hydroxy lower alkyl) amine oxides comprise one or more selected from bis(2-hydroxyethyl) cocoamine oxide, and bis(2-hydroxyethyl) stearylamine oxide; and
the alkamidopropyl di(lower alkyl) amine oxides comprise cocoamidopropyl dimethyl amine oxide.

37. A composition according to claim 33, wherein the one or more amine oxide surfactants comprise decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germamidopropylamine oxide, isostearamido-propylamine oxide, stearamine oxide, stearamido-propylamine oxide, decylamine oxide, dihydroxyethyl C8-C18 alkoxypropylamine oxide, dihydroxyethyl stearamine oxide, myristamidopropylamine oxide, myristamine oxide, palmitamidopropylamine oxide, palmitamine oxide, undecylenamidopropylamine oxide, and mixtures of any two or more thereof.

38. A composition according to claim 27, wherein the one or more amine oxide surfactants comprise lauramine oxide, myristamine oxide, cocamine oxide, lauramidopropyl amine oxide, myristamidopropyl amine oxide, cocamidopropyl amine oxide and mixtures of any two or more thereof.

39. A composition according to claim 27, wherein the one or more amine oxide surfactants are present in an amount of about 1 to about 5% by weight of the composition.

40. A composition according to claim 27, further comprising one or more electrolytes, wherein the one or more electrolytes optionally comprise sodium chloride.

41. A composition according to claim 27, further comprising one or more liquefied gases wherein the one or more liquefied gases optionally comprises a hydrocarbon.

42. A composition according to claim 27, wherein the pH of the composition is in the range of about 6.0 to about 8.0.

43. A composition according to claim 27, further comprising one or more ingredients selected from oils; natural or synthetic scrub particles; visual effect agents; humectants; cationic conditioning agents; preservatives, colorants, chelating agents, pH adjusters, or a clay-based rheology modifier; or
further comprising one or more ingredients selected from emollients, moisturizers, conditioners, skin conditioners, hair conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, liporegulators, weight-reduction agents, anti-acne agents, antiseborrhoeic agents, anti-ageing agents, anti-wrinkle agents, keratolytic agents, anti-inflammatory agents, anti-acne agents, antibiotics, anti-inflammatory agents, botanical extracts, imidazoles, refreshing agents, cicatrizing agents, vascular-protection agents, agents for the reduction of dandruff, seborrheic dermatitis, or psoriasis, shale oil and derivatives thereof, anti-psoriasis agents, corticosteroids depilating agents, agents for combating hair loss, reducing agents for permanent-waving, reflectants, essential oils and fragrances.

44. A method of manufacturing a personal cleansing composition according to claim 27, the method comprising the steps of:
i) loading an amount of water into a suitable vessel;
ii) adding a HASE polymer, one or more anionic surfactants and one or more amine oxide surfactants sequentially in any order, with sufficient mechanical stirring to ensure that each material is fully dispersed before adding the next; and
iii) continuing mixing until a uniform consistency is achieved.

45. A method according to claim 43, wherein the method comprises the following steps:
i) dissolve one or more anionic surfactants in an amount of water;
ii) add HASE polymer emulsion, mixing until the polymer is substantially dispersed in the one or more anionic surfactants;
iii) add one or more amine oxide surfactants; and
iv) mix until substantially uniform consistency is achieved.

46. A method according to claim 43, wherein any of the surfactant or polymer constituents are pre-mixed with an amount of water in any proportion before being added; and where an equal amount of water is subtracted from the starting an amount of water added in step (i).
